(19) United States
(12) Patent Application Publication
(10) Pub. No.: US 2024/0255392 A1
(43) Pub. Date: Aug. 1, 2024

(54) SYSTEMS AND METHODS FOR CARBON DIOXIDE MEASUREMENT

(71) Applicant: University of North Carolina at Wilmington, Wilmington, NC (US)

(72) Inventors: Phil Bresnahan, Wilmington, NC (US); Michael Tydings, Wilmington, NC (US); Elizabeth Farquhar, Wilmington, NC (US)

(73) Assignee: University of North Carolina at Wilmington, Wilmington, NC (US)

(21) Appl. No.: 18/427,391

(22) Filed: Jan. 30, 2024

Related U.S. Application Data
(60) Provisional application No. 63/441,858, filed on Jan. 30, 2023.

(51) Int. Cl. G01N 1/26; G01N 1/22; G01N 33/18

(52) U.S. Cl. CPC G01N 1/26; G01N 1/2214; G01N 33/18

(57) ABSTRACT

A carbon dioxide (CO₂) measurement system is provided. The system comprises an air-side gas permeation unit and a water side gas permeation unit in operable fluid communication with a housing. The permeations units feed a gas stream within the housing, where the gas stream passes through a CO₂ sensor in fluid communication with the gas stream. A control unit in the housing can control the gas stream and/or at least the sensor such that the system is configured to determine a delta value corresponding to a difference between an air-side CO₂ value and a water-side CO₂ value.

### CROSS-REFERENCE TO RELATED APPLICATIONS

This application claims priority to U.S. Provisional Patent Application No. 63/441,858, filed on Jan. 30, 2023, the contents of which is incorporated by reference herein in its entirety.

### FIELD

The technology described herein generally relates to systems, devices, and methods for measuring carbon dioxide, and more specifically to systems, devices, and methods for measuring and/or monitoring carbon dioxide and related metrics at an air-water interface.

### BACKGROUND

Aquatic environments take up or give off carbon dioxide (CO₂) depending on many environmental factors, and being able to characterize the rate is increasingly valuable in applications such as ocean carbon dioxide removal, aquaculture, and oceanographic research.

For instance, changes or differences in CO₂ levels in aquatic ecosystems are especially valuable to scientists or companies who are trying to determine how much CO₂ is entering or exiting or exiting the water. For example, data or metrics related to CO₂ in aquatic ecosystems can provide valuable information for coastal oceanographers, aquaculturists, organizations trying to determine CO₂ uptake of a seagrass or kelp farm for carbon credits, etc. Further, as will be appreciated, Carbon Dioxide Removal (CDR) is estimated to be a $6.7B industry currently, and projected to be about a $200B industry by the year 2050. Accordingly, Measurement, Reporting, and Verification (MRV) of the aforementioned CDR projects is a critical element in the industry, with few solutions.

Currently, conventional systems or methods for measuring or otherwise monitoring carbon dioxide in aquatic or ocean ecosystems are very expensive and have large and unwieldy instruments that have to incorporate on-board gas standards that perform their own calibrations to produce accurate data or alternatively, less expensive instruments are also known, however, are considerably less accurate. Accordingly, aspects of the present technology provide improved systems and methods for measuring and/or monitoring carbon dioxide and related metrics at an air-water interface within compact systems.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are directed towards systems and methods for measuring and/or monitoring CO₂ at or near an air-water interface. According to some aspects, systems and methods described herein can directly measure the difference in CO₂ related values by providing air-side measurements and water-side measurements, in some instances simultaneously.

According to some embodiments, a carbon dioxide (CO₂) measurement system comprises a housing and an air-side gas permeation unit in operable and/or fluid communication with the housing, and a water-side gas permeation unit in operable and/or fluid communication with the housing. The gas permeation units can feed a gas stream, a portion of which is in the housing such that gas stream is in fluid communication with the air-side gas permeation unit and the water-side gas permeation unit. The system further comprises at least one CO₂ sensor within the housing in fluid communication with the gas stream, and a control unit within the housing can control at least the gas stream and the CO₂ sensor, such that the system is configured to determine a delta value corresponding to a difference between an air-side CO₂ value and a water-side CO₂ value.

According to some further embodiments methods of measuring CO₂ in an aquatic environment is provided. An air-side CO₂ input into a gas stream of a CO₂ measurement system can be received, and a water-side CO₂ into the gas stream of the CO₂ measurement system can be received. Based on the received inputs from air-side and water-side gas permeations units, a delta value can be determined corresponding to a difference between an air-side CO₂ value and a water-side CO₂ value.

Additional objects, advantages, and novel features of the invention will be set forth in part in the description which follows, and in part will become apparent to those skilled in the art upon examination of the following, or can be learned by practice of the technology.

### BRIEF DESCRIPTION OF THE DRAWINGS

Having described the presently disclosed subject matter in general terms, aspects of the technology presented herein are described in detail below with reference to the accompanying drawing figures, which are not necessarily drawn to scale, wherein:

FIG. 1 illustrates an example CO₂ measurement and/or monitoring system, in accordance with some aspects of the technology described herein;

FIG. 2 illustrates an example wiring diagram of a CO₂ measurement and/or monitoring system, in accordance with some aspects of the technology described herein;

FIG. 3 illustrates a schematic diagram of an example CO₂ measurement and/or monitoring system, in accordance with some aspects of the technology described herein;

FIG. 4 illustrates a diagram of an example CO₂ measurement and/or monitoring system, in accordance with some aspects of the technology described herein; and

FIG. 5 is a block diagram of an example computing environment and/or device architecture in which some implementations of the present technology may be employed.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7. All ranges disclosed herein are also to be considered to include the end points of the range, unless expressly stated otherwise. For example, a range of “between 5 and 10” or “5 to 10” or “5-10” should generally be considered to include the end points 5 and 10. Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the claims.

In an embodiment, a carbon dioxide (CO₂) measurement system comprises a housing, which can house or partially house one or more modules, devices, units, tubes, conduits, etc. In an embodiment, a device and/or system can comprise an air-side gas permeation unit, which can be in operable and/or fluid communication with the housing. In some aspects the housing can contain a contain a portion of the air-side gas permeation unit. In some embodiments, a CO₂ measurement and/or monitoring system can comprise a water-side gas permeation unit in operable and/or fluid communication with the housing. In some aspects the housing can contain a contain a portion of the water-side gas permeation unit. The gas permeation units (e.g. at least one of the air-side gas permeation unit(s) and the water-side gas permeation unit(s)) can feed a gas stream, at least a portion of which is in the housing such that gas stream is in fluid communication with the air-side gas permeation unit and/or the water-side gas permeation unit. As will be appreciated the gas stream can be implemented and/or configured in any manner non inconsistent with the technical objectives of the present technology. In some aspects, a gas permeation unit comprises a permeation cell and/or membrane. In some embodiments a CO₂ measurement and/or monitoring system comprises a CO₂ sensor that can be in operable communication (e.g. fluid communication) with at least a portion of a gas stream. In some embodiments, a CO₂ measurement and/or monitoring system comprises a control unit which can be contained in the housing. In some instances, at least one of the CO₂ sensor and/or a control unit are configured to determine a delta value (i.e. a CO₂ delta, difference, change, value) which corresponds to a difference between an air-side CO₂ value and a water-side CO₂ value. As will be appreciated any value(s) or value unit(s) not inconsistent with the technical objectives of the present technology may be used. Further, in some aspects, the delta value (CO₂ delta value) can correspond to or be related to a flux of CO₂, for example a flux of CO₂ with respect to the system or a flux of CO₂ at an air-water interface where a measurement and/or monitoring system can be employed. In some aspects, the system or at least one of a CO₂ sensor and/or a control unit and/or a permeation unit is configured to measure or otherwise determine an air-side CO₂ value and/or a water-side CO₂ value. In some embodiments an air-side CO₂ value and/or a water-side CO₂ value is at least one of a pCO₂ value and/or an xCO₂ value. In some embodiments, a system can have one or more gas streams, and for instance can comprise an air-side gas stream, and/or a water-side gas stream, and/or a combined gas stream, e.g. the combined gas stream combining (e.g. via a valve) an air-side gas stream and a water-side gas stream. In some embodiments, a combined gas stream can be split (e.g. via a valve) into an air-side gas stream and a water-side gas stream. In some embodiments, a system comprises at least one of a desiccant chamber, a pump, a thermometer (e.g. air-side thermometer, water-side thermometer), a microcontroller, a data logger, a wireless communication component or device. In some embodiments, any one of the system components can be in operable communication with a control unit.

According to embodiments of the technology described herein, systems, devices, and methods are provided for measuring and/or monitoring carbon dioxide (CO₂) and/or CO₂ related metrics at an air-water interface. In some instances, a CO₂ measurement and/or monitoring device or system can include at least one sensing device or sensor (e.g. CO₂ sensing device) and comprise a plurality of physical components and software, more specifically firmware including control aspects, configured to measure and/or monitor air and/or water CO₂ near or at an air-water interface. In some instances, a CO₂ sensing device can measure and/or monitor a mole fraction of air and/or water CO₂ (i.e. xCO₂), a partial pressure of air and/or water CO₂ (i.e. pCO₂), or a concentration of air and/or water CO₂ (e.g. molar concentration).

Accordingly, systems, devices, and methods are provided for implementing a relatively lower cost (compared to conventional systems or instruments) CO₂ monitoring and/or measurement system and/or device which is configured to monitor and/or measure both air and water CO₂, for instance xCO₂ and/or pCO₂, and further provide monitoring and/or measurement near or at an air-water interface. As will be appreciated devices, systems, and methods described herein can be configured to measure air CO₂, water CO₂, or both simultaneously or in series. As will be appreciated, air CO₂ concentrations are relatively stable, enabling an air-based validation of the measurement and collection of an accurate delta (Δ) value or difference between air-and water-side CO₂ at or around an air-water interface.

According to some embodiments, a ΔCO₂ measurement system (also referred to herein as a ΔCO₂ measurement device, or alternatively monitoring device and/or system) is provided. As will be appreciated, in some embodiments a ΔCO₂ measurement system can monitor and/or measure a ΔxCO₂ and/or ΔpCO₂. As described herein, ΔCO₂ may in some instances refer to at least one of ΔxCO₂ and ΔpCO₂.

According to some further embodiments, a ΔpCO₂ measurement system and/or device is provided. In some instances, the ΔpCO₂ measurement system can measure and/or determine, or otherwise monitor both an air-side pCO₂ value (partial pressure of CO₂ in air) and a water-side pCO₂ value (partial pressure of CO₂ in water), either simultaneously or individually. Without intending to be bound by theory, in some aspects ΔpCO₂ can be directly relatable and/or correspond to the flux of CO₂ into/out of water from/to the air. Accordingly, some aspects of the present technology are directed towards determining the flux of CO₂ at an air-water interface or in an aquatic environment.

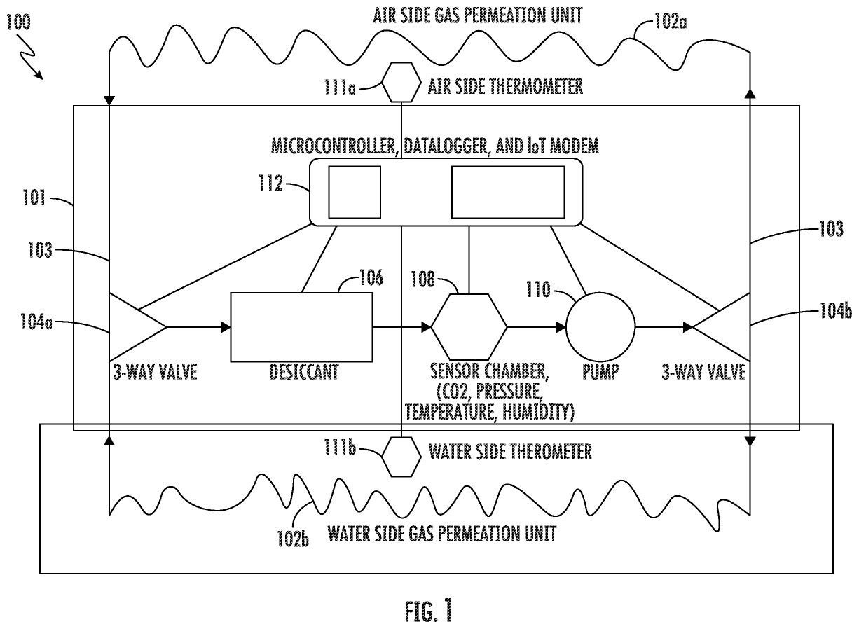

Turning now to the figures, FIG. 1 illustrates an example CO₂ measurement and/or monitoring system, in accordance with some aspects of the present technology. FIG. 1 is discussed herein with respect to the measurement and/or monitoring of a ΔpCO₂ at an air water interface, however, it will be appreciated that various aspects can also be applicable to the measurement and/or monitoring of a ΔxCO₂, and/or additionally other CO₂ or related metrics at an air-water interface or in an aquatic environment.

FIG. 1 generally depicts aspects of a CO₂ measurement system, and more particularly an example flow diagram of a ΔpCO₂ system 100. ΔpCO₂ system 100 can include one or more gas permeation units 102a, 102b, for example air-side gas permeation unit 102a and water side gas permeation unit 102b. The air-side gas permeation unit 102a and the water side gas permeation unit 102b are configured to allow CO₂ to diffuse from the environment (e.g. air or water) into a gas stream 103 of the ΔpCO₂ system 100. The gas stream 103 can move from each of the gas permeation units 102a, 102b, to a three-way valve 104a, within a waterproof housing 101, that is configured to direct the gas stream(s) from the gas permeation units to or through one or more downstream units of the ΔpCO₂ system 100. As depicted, ΔpCO₂ system 100 can further comprise a plurality of components, such as a desiccant chamber 106, a sensor chamber 108, a pump 110, and another, or second, three-way valve 104b.

As shown in FIG. 1, the CO₂ in the system from each one of the gas permeation units moves through the system to three-way valve 104a, to desiccant chamber 106, to sensor chamber 108, to pump 110, to another three-way valve 104b, before returning to one of gas permeation units 102a, 102b. Among the features not shown, sensor chamber 108 can include one or more sensors, for example a CO₂ sensor, a pressure sensor, a temperature sensor, and/or a humidity sensor. As will be appreciated, any combination of sensors may be included in sensor chamber 108.

ΔpCO₂ system 100 can further include one or more control units 112 that are in operable communication with any components and/or modules of the system 100. Accordingly, any or all of the components described above, as well as one or more temperature measurement components 111a, 111b, for example air-side temperature measurement component 111a (e.g. air-side thermometer) and water-side temperature measurement component 111b (e.g. water-side thermometer), can be in operable or digital communication with control unit 112. Control unit 112 can include one or more modules, such as microcontroller 114, data logger 116, and modem 118. Modules 114, 116, and 118 can be configured or implemented for various control and/or measurement aspects of any components or other modules of system 100. According to some aspects, microcontroller 114 can run firmware configured to control one or more of the system components (e.g. three-way valves 104a, 104b, or pump 110) as well as to record and/or process data generated by or input into the system, for instance microcontroller 114 can record or process data from any of the sensors in sensor chamber 108 (e.g. CO₂ sensor, pressure sensor, temperature sensor, and/or humidity sensor) as well as temperature measurement components (or sensors) 111a, 111b. In some instances, control unit 112 includes data logger 116 to store any of the collected data from the system (which can be implemented as storage media, such as digital storage media) and further control unit 112 can include a modem 118 (e.g. implemented as an IoT modem, for example a cellular data transfer unit) to transmit data to another system or a database, such as a cloud database. As will be appreciated, data can alternatively be transferred directly from data logger 116 to another system or database.

Among aspects not illustrated, CO₂ system 100 can further include a rechargeable battery or battery system to provide power for operation, and additionally, one or more solar panel units may be integrated into CO₂ system 100 for providing direct power or for charging a battery or battery system while CO₂ system 100 is deployed. As will be appreciated, control unit 112 can operate and/or manage the battery system and solar panel units. Additionally, CO₂ system 100 can also include or integrate any number of other sensors, such as anemometers and/or current meters for the measurement of wind and water current speeds.

According to some embodiments, CO₂ system 100 can be directly or remotely operated, or alternatively operate autonomously, i.e. CO₂ system 100 can be deployed into an aquatic environment to gather, measure, determine, record, and transmit data without the need for any human intervention for a period of time. Further, CO₂ system 100 can be deployed on or integrated with, for example, aquatic buoys, small vessels such as kayaks and canoes, and autonomous or crewed research vessels.

According to some even further embodiments, once a set of data has been collected and recorded (such as data relating to air-and water-side CO₂, temperature, pressure, humidity, etc.) the data or data can be processed via one or more algorithms, and/or with other data, for example third party data or information such as wind speed data which can be accessed via auxiliary databases. In some instances, CO₂ system 100 can be configured to access one or more auxiliary databases, such as environmental prediction databases. As will be appreciated, CO₂ system 100 can be in communication with one or more additional user devices which can carry out any number of processing steps not handled by CO₂ system 100 as illustrated in FIG. 1. Accordingly, CO₂ system 100 and other integrated components or systems can determine a carbon dioxide flux (e.g. CO₂ flux or flux value) that indicates the rate of transfer of CO₂ into or out of an aquatic environment. As described herein, the incorporation of an anemometer and/or a current meter into CO₂ system 100 can alternatively enable a direct determination of CO₂ flux without the need to auxiliary data.

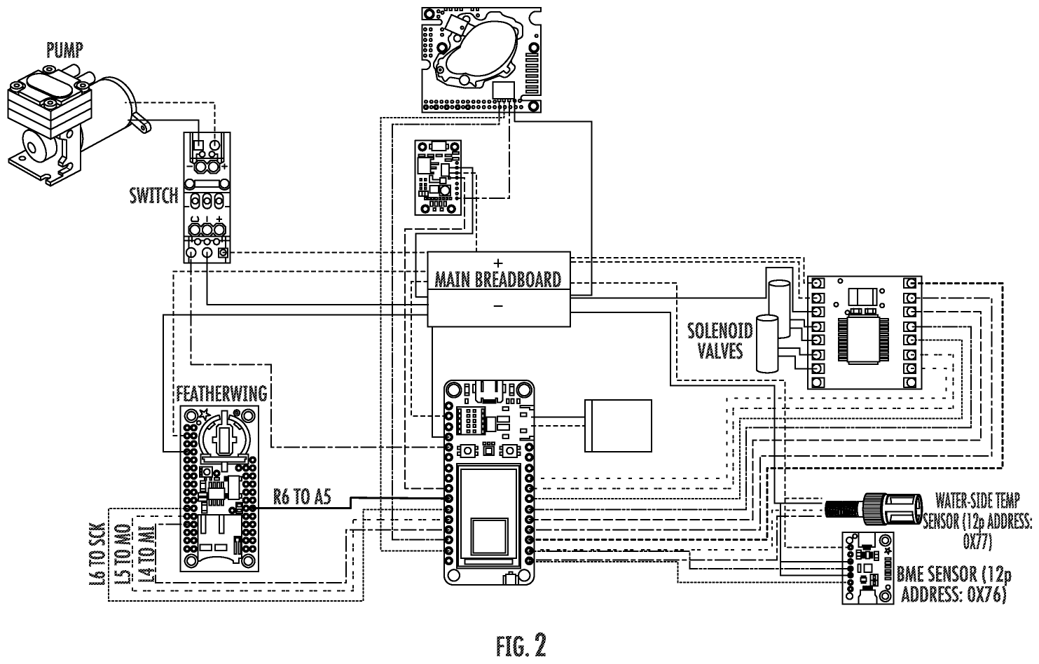

Referring briefly to FIG. 2, FIG. 2 illustrates an example wiring diagram for various components of a CO₂ measurement and/or monitoring system (e.g. ΔpCO₂ system 100 of FIG. 1). Accordingly, FIG. 2 illustrates an example conceptual block diagram of sensors and other electrical components of a CO₂ measurement and/or monitoring system. As will be appreciated, FIG. 2 is illustrative and additional or fewer components and connections may be desired or otherwise required.

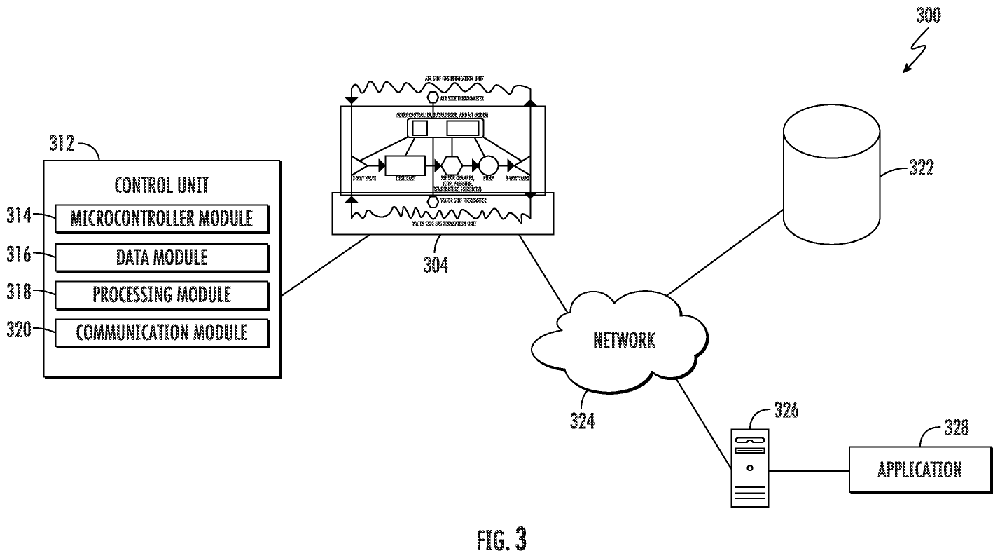

FIG. 3 is a schematic depicting various aspects of a CO₂ measurement system 300, in accordance with various embodiments of the present disclosure. The CO₂ measurement system 300 can include CO₂ measurement device 304 and can further include or integrate a plurality of engines, modules, or components that make up a control unit stack 312 (alternatively referred to as a device operation stack), which can include but is not limited to: a microcontroller module 314, a data logger and/or storage module 316, a processing module 318, and communication module 320. In some instances, communication module 320 can further incorporate a connectivity module or component. Among other components not shown, CO₂ measurement system 300 can operate in conjunction with other software applications and systems as well as computing hardware can include a user interface and/or user input components for device operation and associated engines and/or modules for their connectivity and operation.

CO₂ measurement system 300 can comprise a content repository 322, which can device 304 or one or more associated or integrated modules (e.g. modules 214-220). Content repository 322 can be a local or remote storage device that can contain or host data associated with CO₂ measurement system 300 or one or more related applications 104. In some instances, content repository 322 can store or host auxiliary data for use with CO₂ measurement device 304 or host one or more sets of data generated by CO₂ measurement device 304. In some instances, data module 316 can store and/or organize one or more data sets associated with CO₂ measurement device 304. As will be appreciated, any number of modules for CO₂ measurement device 304 can run locally on the device or run remotely, as a part of a distributed system (e.g. distributed operation stack).

CO₂ measurement system 300 contemplates both wired and wireless systems, and in one embodiments, CO₂ measurement device 304 can be implemented within a wired and/or wireless network, and further be connected to one or more other computing devices via network 324. In some embodiments, CO₂ measurement device 304 may be in operable or digital communication with one or more additional computing devices or servers 326. In some instances, CO₂ measurement device 304 and/or any one of the modules of control unit stack 312 can send and/or receive data or information to/from another application 328 running on the one or more additional computing devices or servers 326.

Microcontroller module 314 is generally responsible for the operation of the components of CO₂ measurement device 304, such as one or more valves, desiccant chambers, sensors, pumps (e.g. 104a, 104b, 106, 108, 110 of FIG. 1), or other measurement units (e.g. temperature measurement units 111a, 111b of FIG. 1). Microcontroller module 314 can initiate any one of the units of CO₂ measurement device 304 and provide control operations independently and/or based on any input or input signal (digital or physical) to the device. Data module 316 is generally responsible for handling and storing data (or data sets) and/or input to the CO₂ measurement device 304. In some instances, data module 316 can organize or transform data or data sets, or further store such data or data sets prior to transmission or store data onboard the CO₂ measurement device 304. Processing module 318 is generally responsible for processing the data or data sets collected and/or stored by the data module, for instances to apply one or more algorithms and/or functions to the collected and/or stored data. In some embodiments, processing module 318 can be instantiated as another application or a part of another application or process running on another device and/or server (e.g. device 326). Communication module 320 is generally responsible for transmitting data or one or more sets of data from CO₂ measurement device 304 to another user device or system or alternatively another data storage (e.g. data storage 322).

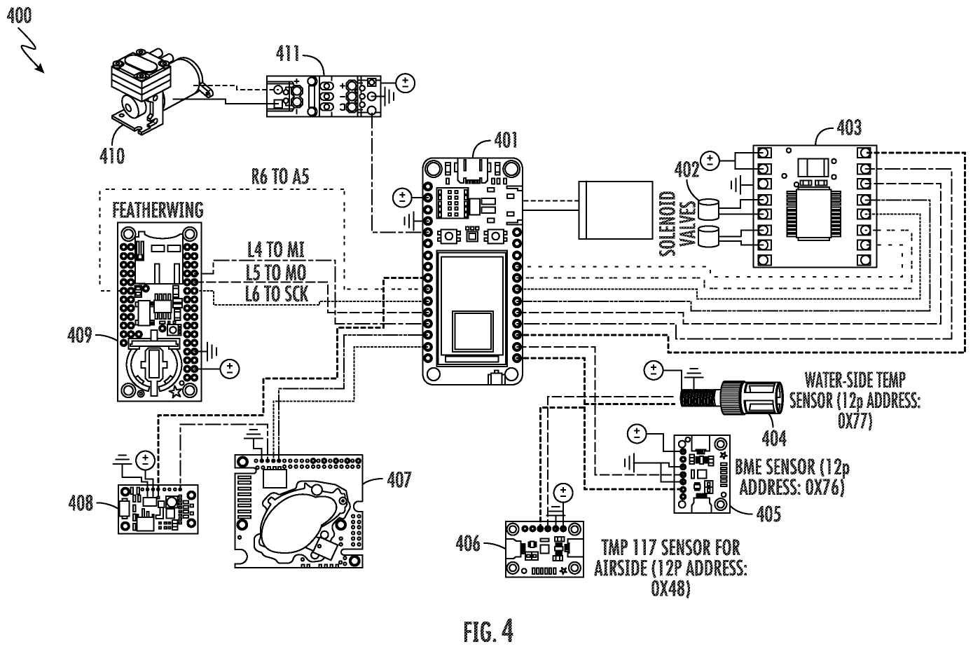

Referring now to FIG. 4, a diagram of an example CO₂ measurement and/or monitoring system 400 is illustrated, in accordance with some aspects of the technology described herein. In some aspects, FIG. 4 illustrates an example block diagram of sensors, wiring, and other electrical components of a CO₂ measurement and/or monitoring system. CO₂ measurement and/or monitoring system 400 can include microcontroller 401, one or more valves, such as solenoid valves 402, a motor driver 403 which can be configured to control one or more valves, a water-side temperature sensor 404, a pressure, and/or temperature, and/or humidity sensor for monitoring and/or measuring an internal gas stream, an air-side temperature sensor 406, a CO₂ sensor 407, a power boost circuit 408, a data logger board 409, a gas pump 410, and a switch 411 which can be configured for gas pump control. As will be appreciated, FIG. 4 is illustrative and additional or fewer components and connections may be desired or otherwise required.

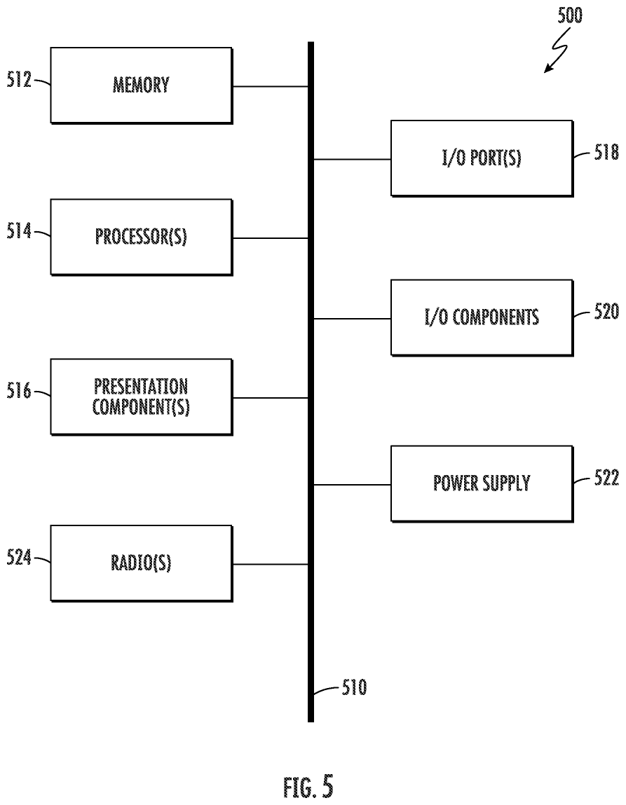

FIG. 5 provides an illustrative example operating environment for implementing embodiments of the present invention and can be designated generally as computing device 500. As will be appreciated, an example operating environment for which embodiments of the present technology may be implemented is described to provide a context for various aspects of the technology. Computing device 500 is merely one example of a suitable computing environment and is intended to suggest any limitation as to the scope of use or functionality of the invention. Neither should the computing device 500 be interpreted as having any dependency or requirement relating to any one or combination of components illustrated.

Embodiments of the invention can be described in the general context of computer code or machine-useable instructions, including computer-executable instructions such as program modules, being executed by a computer or other machine (virtual or otherwise), such as a smartphone or other handheld device. Generally, program modules, or engines, including routines, programs, objects, components, data structures etc., refer to code that perform particular tasks or implement particular abstract data types.

Embodiments of the invention can be practiced in a variety of system configurations, including hand-held devices, consumer electronics, general-purpose computers, more specialized computing devices, etc. Embodiments of the invention can also be practiced in distributed computing environments where tasks are performed by remote-processing devices that are linked through a communications network.

With reference to FIG. 5, computing device 500 includes a bus 510 that directly or indirectly couples the following devices: memory 512, one or more processors 514, one or more presentation components 516, input/output ports 518, input/output components 520, and an illustrative power supply 522. In some embodiments, devices described herein utilize wired and rechargeable batteries and power supplies. Bus 510 represents what can be one or more busses (such as an address bus, data bus or combination thereof). Although the various blocks of FIG. 5 are shown with clearly delineated lines for the sake of clarity, in reality, such delineations are not so clear and these lines can overlap. For example, one can consider a presentation component such as a display device to be an I/O component as well. Also, processors generally have memory in the form of cache. It is recognized that such is the nature of the art, and reiterate that the diagram of FIG. 5 is merely illustrative of an example computing device that can be used in connection with one or more embodiments of the present disclosure. Distinction is not made between such categories as “workstation,” “server,” “laptop,” “hand-held device,” etc., as all are contemplated within the scope of FIG. 5 and reference to “computing device.”

Computing device 500 typically includes a variety of computer-readable media. Computer-readable media can be any available media that can be accessed by computing device 500, and includes both volatile and non-volatile media, removable and non-removable media. By way of example, and not limitation, computer-readable media can comprise computer storage media and communication media.

Computer storage media (or digital storage media) can include volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 500. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner at to encode information in the signal. By way of example, and not limitation, communication media includes wired media such as a wired network or direct-wired connection, and wireless media such as acoustic, RF, infrared, NFC, Bluetooth, LTE, and other wireless media. Combinations of any of the above should also be included within the scope of computer-readable media.

Memory 512 includes computer storage media in the form of volatile and/or non-volatile memory. As depicted, memory 512 includes instructions that when executed by processor(s) 514 are configured to cause the computing device to perform any of the operations described herein, in reference to the above discussed figures, or to implement any program modules described herein. The memory can be removable, non-removable, or a combination thereof. Illustrative hardware devices include solid-state memory, hard drives, optical-disc drives, etc. Computing device 500 includes one or more processors that read data from various entities such as memory 512 or I/O components 520. Presentation component(s) 516 present data indications to a user or other device. Illustrative presentation components include a display device, speaker, printing component, vibrating component, etc.

I/O ports 518 allow computing device 500 to be logically coupled to other devices including I/O components 520, some of which can be built in. Illustrative components include a microphone, joystick, game pad, satellite dish, scanner, printer, wireless device, battery, etc. Additionally, computing device 500 can include one or more radios 524 which can wirelessly transmit and/or receive information and/or data to/from a network or wireless network. In some instances, one or more radios can transmit and receive wireless signals.

Various operations have been described as multiple discrete operations in a manner that can be illustrative and helpful in understanding the embodiments presented. However, the order of the description should not be construed as to imply that these operations are necessarily order dependent. Further, descriptions of entities and/or modules should not be construed as requiring the modules or that the modules be separate and/or perform separate operations. In various embodiments, illustrated and/or described operations, entities, data, modules, components, and/or engines may be merged, broken into parts and/or further sub-parts, and/or omitted.

Many variations can be made to the illustrated embodiment of the present invention without departing from the scope of the present invention. Such modifications are within the scope of the present invention. Embodiments presented herein have been described in relation to particular embodiments which are intended in all respects to be illustrative rather than restrictive. Alternative embodiments and modifications would be readily apparent to one of ordinary skill in the art, but would not depart from the scope of the present invention.

From the foregoing, it will be seen that this invention is one well adapted to attain all ends and objects hereinabove set forth together with the other advantages which are obvious and which are inherent to the structure. It will be understood that certain features and sub combinations are of utility and can be employed without reference to other features and sub combinations. This is contemplated by and is within the scope of the invention.

In the preceding detailed description, reference is made to the accompanying drawings which form a part hereof wherein like numerals designate like parts throughout, and in which is shown, by way of illustration, embodiments that can be practiced. It is to be understood that other embodiments can be utilized and structural or logical changes can be made without departing from the scope of the present disclosure. Therefore, the preceding detailed description is not to be taken in the limiting sense, and the scope of embodiments is defined by the appended claims and their equivalents.

Various operations have been described as multiple discrete operations, in turn, in a manner that is most helpful in understanding the illustrative embodiments; however, the order of description should not be construed as to imply that these operations are necessarily order dependent. In particular, these operations need not be performed in the order of presentation. Further, descriptions of operations as separate operations should not be construed as requiring that the operations be necessarily performed independently and/or by separate entities. Descriptions of entities and/or modules as separate modules should likewise not be construed as requiring that the modules be separate and/or perform separate operations. In various embodiments, illustrated and/or described operations, entities, data, and/or modules can be merged, broken into further sub-parts, and/or omitted.

The terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth. Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Although the foregoing subject matter has been described in some detail by way of illustration and example for purposes of clarity of understanding, it will be understood by those skilled in the art that certain changes and modifications can be practiced within the scope of the appended claims. Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below.

Certain features and subcombinations are of utility and can be employed without reference to other features and subcombinations and are contemplated within the scope of the claims.

1. A carbon dioxide (CO₂) measurement system comprising:
a housing;
an air-side gas permeation unit in operable communication with the housing;
a water-side gas permeation unit in operable communication with the housing;
a gas stream, wherein at least a portion of the gas stream is within the housing and the gas stream is in fluid communication with the air-side gas permeation unit and the water-side gas permeation unit;
at least one CO₂ sensor within the housing in fluid communication with the gas stream; and
a control unit within the housing, wherein at least one of the CO₂ sensor and the control unit are configured to determine a delta value corresponding to a difference between an air-side CO₂ value and a water-side CO₂ value.

2. The system of claim 1, wherein the delta value is related to a flux of CO₂.

3. The system of claim 1, wherein the air-side CO₂ value is at least one of a pCO₂ value and a xCO₂ value.

4. The system of claim 1, wherein the water-side CO₂ value is at least one of a pCO₂ value and a xCO₂ value.

5. The system of claim 1, further comprising a first valve configured to combine an air-side gas stream and a water-side gas stream into a combined gas stream.

6. The system of claim 1, further comprising a second valve configured to split a combined gas stream into an air-side gas stream and a water-side gas stream.

7. The system of claim 1, wherein the CO₂ sensor is contained within a sensor chamber, the sensor chamber further comprising at least one of a pressure sensor, a temperature sensor, and a humidity sensor.

8. The system of claim 1 further comprising a desiccant chamber.

9. The system of claim 1 further comprising a pump.

10. The system of claim 1 further comprising at least one air-side thermometer and/or at least one water-side thermometer.

11. The system of claim 1, wherein the control unit is in operable communication with at least one of a first valve, a second valve, a desiccant chamber, a sensor chamber, an air-side thermometer, and a water-side thermometer.

12. The system of claim 1, wherein the delta value is determined based at least in part on a retrieved data set and/or algorithm.

13. The system of claim 1, wherein the control unit comprises a microcontroller.

14. The system of claim 1, wherein the control unit comprises a data logger.

15. The system of claim 1, wherein the control unit comprises a wireless communication component.

16. A method of measuring CO₂ in an aquatic environment, the method comprising:
receiving, by a CO₂ measurement system of claim 1, an air-side CO₂ input into a gas stream of the CO₂ measurement system;
receiving, by the CO₂ measurement system, a water-side CO₂ input into the gas stream of the CO₂ measurement system; and
determining a delta value corresponding to a difference between an air-side CO₂ value and a water-side CO₂ value.

17. The method of claim 16 further comprising generating a data set corresponding to one or more delta values determined over a period of time.

18. The method of claim 17, further comprising transmitting the dataset to a user device and/or data store.

19. The method of claim 16, further comprising determining an air-side CO₂ value and/or a water-side CO₂ value, each value being at least one of a pCO₂ value and a xCO₂ value.

20. The method of claim 19, wherein the air-side CO₂ value and/or a water-side CO₂ value are determined simultaneously.
